(19) United States
(12) Patent Application Publication
(10) Pub. No.: US 2016/0183922 A1
(43) Pub. Date: Jun. 30, 2016

(54) APPARATUS AND METHOD FOR PLANAR IMAGE SYNCHRONIZATION

(71) Applicant: Oreon Technologies Inc., Irvington, NY (US)

(72) Inventors: Juan Oscar Estrada Arredondo, Stanton, CA (US); Howard F. Fidel, Irvington, NY (US); Raul F. Gutierrez, Fort Lee, NJ (US)

(21) Appl. No.: 14/936,608

(22) Filed: Nov. 9, 2015

Related U.S. Application Data
(60) Provisional application No. 62/076,702, filed on Nov. 7, 2014.

(51) Int. Cl. A61B 8/00; A61B 6/03; A61B 5/055; A61B 5/00; A61B 8/12

(52) U.S. Cl. CPC A61B 8/463; A61B 5/0035; A61B 8/12; A61B 5/055; A61B 6/032; A61B 2562/0219

(57) ABSTRACT

A system and method and for acquiring and displaying images from different image modalities where both image sets are acquired in a sequential planar fashion and can be displayed one slice at a time on a computer screen. The operator can locate an anatomical target, such as the base of the prostate, in this image sequence. On the same screen, a live transverse ultrasound image can be acquired and the anatomical target located in the transverse image. The operator can reposition the transducer while acquiring images at intervals. A sensor detects the transducer position so the locations of the planes can be identified. Any plane can be selected from either image displayed, and the image will track in the alternate modality. The desired slice can be displayed on both images, allowing the operator to follow both images and identify pathology that may not be present in one of the images.

This application claims priority from and the benefit of provisional patent application Ser. No. 62/076,702 filed on Nov. 7, 2015, the entire contents of which are incorporated herein by reference, for all purposes.

### BACKGROUND OF THE DISCLOSURE

1. Field of the Disclosure

The present disclosure relates generally to image acquisition and display systems. More particularly, it relates to displaying multimodality images that may be used for targeting interventional therapies. Still more particularly this invention relates to displaying a template grid system for use with trans-rectal ultrasound imaging probes in biopsy, brachytherapy and cryosurgery for cancerous prostate and related surgeries and displaying them in the same anatomical locations as on a reference MRI image as an ultrasound image, where the ultrasound transducer has sensors embedded in it to detect the position of the transducer.

2. Description of the Related Art

As illustrated in FIG. 1 of U.S. Pat. No. 6,422,997, and in FIG. 1 of United States Patent Publication 20040152986 (the entire contents of which are incorporated by reference herein), brachytherapy is performed with the patient in the lithotomy position, using an ultrasound imaging probe placed in the rectum to monitor seed placement. A template grid arrangement or guide plate having openings at predefined positions of a coordinate grid thereon, of the general type illustrated in FIG. 3 of U.S. Pat. No. 6,387,034, which is kept in precise linear orientation with the ultrasound probe, must be accurately oriented adjacent the perineum in relation to the prostate, and locked in position throughout the procedure to achieve optimum seed placement. A cradle of the type illustrated in FIG. 17 of U.S. Pat. No. 6,752,753 is helpful in this regard. Precise and reproducible orientation and positioning of the ultrasound imaging probe in the rectum is a key element in both the calculations required for determining the number and distribution of radioactive seeds required for treatment and their subsequent placement using preloaded needles guided by the peritoneal template and real time ultrasound imaging. The content of these patents, and those mentioned in the following paragraphs, are incorporated herein by reference, in their entireties, for all purposes.

U.S. Pat. Nos. 8,768,101; 8,532,352; 8,525,852 and 8,447,137, provide some advantages of the approach disclosed below, but are directed to the very different method of image fusion.

### SUMMARY OF THE DISCLOSURE

It is an object of the disclosure to provide an apparatus and a method for displaying diagnostic medical images produced by two different modalities, which allows for comparison of the diagnostic information in both images.

It is a further object to synchronize the display of both images so the same imaging planes are viewed simultaneously.

An additional object of the disclosure is to overlay the template grid image on both image modalities.

These objects and others are achieved in accordance with the disclosure by providing sensors to determine transducer position of the ultrasound transducer.

An embodiment of the invention is directed to an apparatus for displaying images of an organ within the body, comprising an ultrasonic imaging system having at least one display screen; an ultrasonic transducer associated with the ultrasonic imaging system, the ultrasonic transducer being movable to acquire first images of first successive slices of the organ for display by the at least one display screen of the imaging system; a position sensor associated with the ultrasonic transducer for sensing the position of the ultrasonic transducer when the first images of the first successive slices are acquired; and an input for receiving second images of second successive slices of the organ generated by a second imaging system other than an ultrasonic imaging system, the second successive slices of the organ being sequentially displayed on the at least one display screen; wherein one of the first successive images and one of the second successive images, each corresponding to a same slice of the organ, are simultaneously displayed on the at least one display screen.

The input for receiving second images includes an image data acquisition apparatus. The image data acquisition apparatus acquires data from an image data storage medium. The image storage medium is selected from the group consisting of a CD, a DVD, a magnetic disk drive and a flash memory. The image data acquisition apparatus can acquire data from a DICOM device.

The second imaging system can be an MRI system, an x-ray imaging system or a computerized axial tomography imaging system.

The position sensor can be an accelerometer, and position of the ultrasonic transducer is determined by integrating signals from the accelerometer.

In general, the disclosure is also directed to an apparatus for displaying images of an organ within a body comprising: at least one display screen for displaying an image of a first slice of an organ obtained using a first imaging modality; and the at least one display screen also displaying an image of a second slice of an organ obtained using a second imaging modality; wherein the first slice and the second slice are substantially the same slice of the organ.

The first slice is one of a first series of successive slices obtained by the first modality and the second slice is one of a second series of slices obtained by the second modality. The first slice and successive ones of the series of slices obtained by the second modality are displayed on the at least one display screen.

The first imaging modality can be ultrasound. The second imaging modality is MRI can be MRI, x-ray imaging or computerized axial tomography imaging.

The first series of successive slices obtained by the first modality and the second series of slices obtained by the second modality are perpendicular to substantially the same axis.

The disclosure is also directed to a method for displaying images of an organ within the body, comprising: displaying on at least one display screen, an image of a first slice of an organ obtained using a first imaging modality; and displaying on the at least one display screen, an image of a second slice of an organ obtained using a second imaging modality; wherein the first slice and the second slice are substantially the same slice of the organ.

The first slice is one of a first series of successive slices obtained by the first modality and the second slice is one of a second series of slices obtained by the second modality.

The first series of successive slices obtained by the first modality and the second series of slices obtained by the second modality are perpendicular to substantially the same axis.

The method can further comprise, when he first modality is ultrasonic imaging, using a movable ultrasonic transducer associated with the ultrasonic imaging system to acquire the first images of first successive slices of the organ for display by the at least one display screen of the imaging system; and acquiring a position signal representative of position of the sensor associated with the ultrasonic transducer to sense the position of the ultrasonic transducer when the first images of the first successive slices are acquired.

The disclosure is also directed to a computer readable non-transitory storage medium storing instructions of a computer program which when executed by a computer system results in performance of steps of the method for imaging described herein.

### BRIEF DESCRIPTION OF THE DRAWINGS

The foregoing aspects and other features of the present invention are explained in the following description, taken in connection with the accompanying drawing, wherein:

FIG. 1 is a plan view of a brachytherapy template or guide plate in accordance with the invention.

FIG. 2 shows a template grid over both an MRI image and an ultrasound image in the transverse view, and the corresponding ultrasound image in the sagittal view with an indicated target area for biopsy, sampling or treatment delivery.

FIG. 3 is a diagram of a system which can utilize the present disclosure.

FIG. 4 is an alternate grid to be used in place of the sagittal view in FIG. 2 that shows the slice of the image that is displayed in the transverse view.

FIG. 5 is a block diagram of a computer used in the system of FIG. 3.

A component or a feature that is common to more than one drawing is indicated with the same reference number in each of the drawings.

### DESCRIPTION OF THE EMBODIMENT

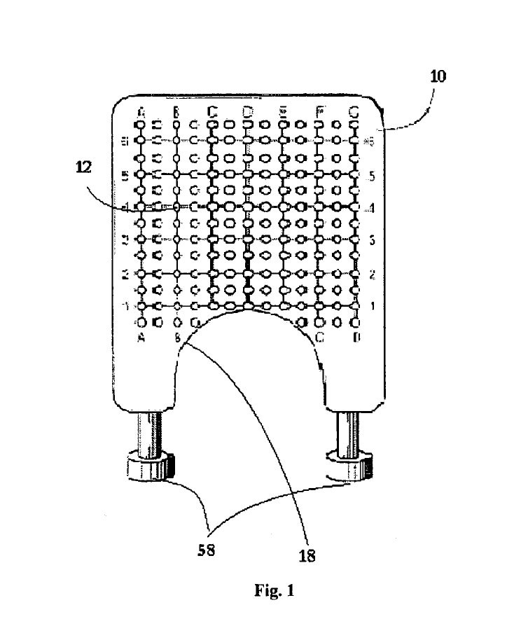

Referring to FIG. 1, there is shown a plan view of a template or guide plate 10 used in the apparatus and method disclosed herein. Although the present disclosure contains a description of specific embodiments shown in the drawings, it should be understood that the other embodiments, in many alternate forms, can be used.

An ultrasound system used to guide the placement of needles or probes in biopsy, brachytherapy and cryosurgery for cancerous prostate and related surgeries has a grid on the screen representing the locations of openings 12 of the template or guide plate 10 used to guide the needles that are inserted into the patient. The first location for the needle to be inserted blinks on the screen. After the system detects that a needle is inserted, the color of that location on the screen changes from green to red, indicating that a needle is already placed at that location. The next location that requires a needle insertion will flash green, until the next needle is inserted. When all the required needles are inserted, all the locations will have turned red.

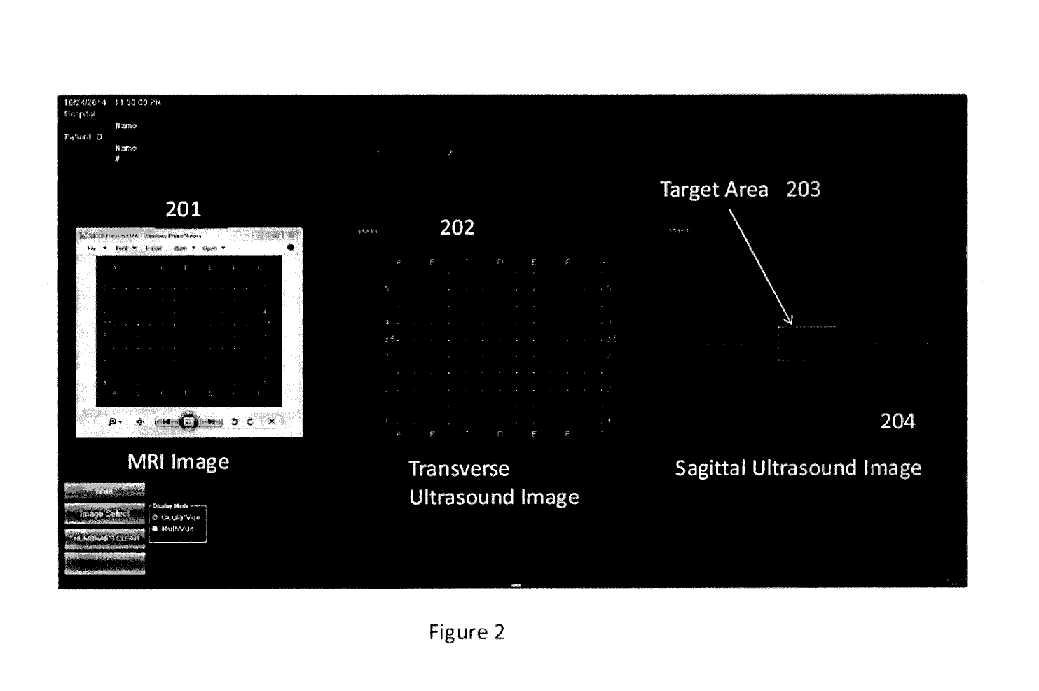

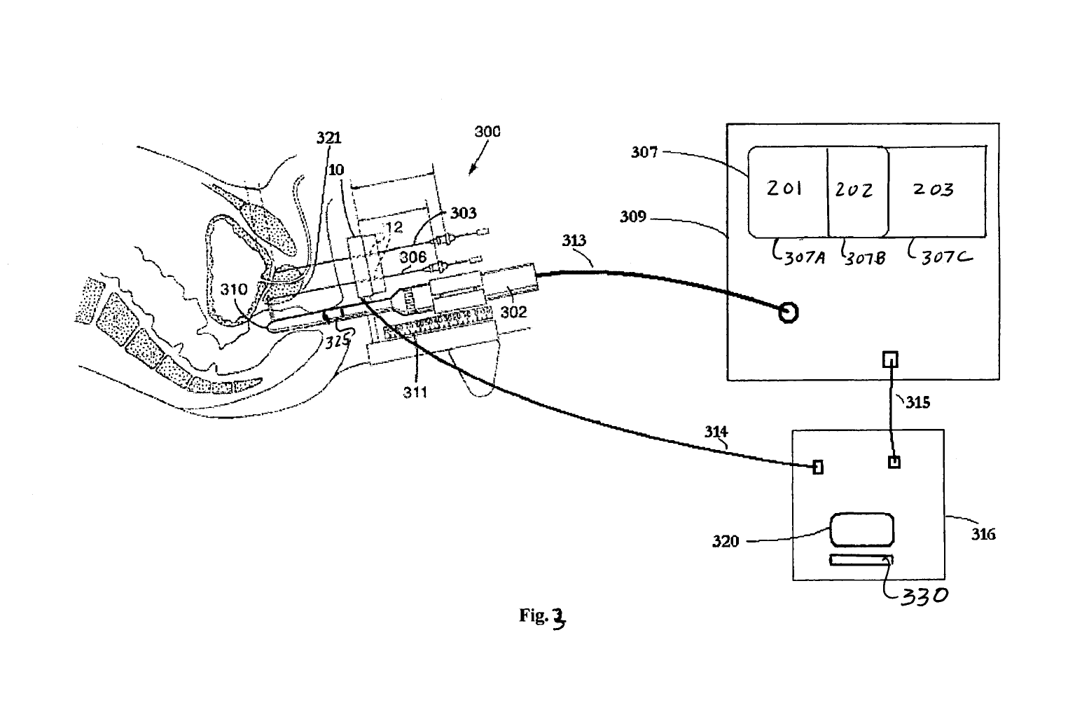

As illustrated in FIG. 2 of U.S. Pat. No. 6,422,997, and in FIG. 1 of United States Patent Publication No. 20040152986 (the entire contents of which are incorporated by reference herein), brachytherapy is performed with the patient in the lithotomy position, using an ultrasound imaging probe placed in the rectum to monitor seed placement. The template or guide plate 10 of FIG. 1 herein, having openings at predefined positions of a coordinate grid thereon, of the general type illustrated in FIG. 3 of U.S. Pat. No. 6,387,034, which is kept in precise linear orientation with the ultrasound probe, must be accurately oriented adjacent the perineum in relation to the prostate, and locked in position throughout the procedure to achieve optimum seed placement. A cradle of the type illustrated in FIG. 17 of U.S. Pat. No. 6,752,753 is helpful in this regard. Precise and reproducible orientation and positioning of the ultrasound imaging probe in the rectum is a key element in both the calculations required for determining the number and distribution of radioactive seeds required for treatment and their subsequent placement using preloaded needles guided by the peritoneal template and real time ultrasound imaging.

As more fully described below with respect to FIG. 2, the ultrasound system used to guide the needles has a grid on the screen representing the locations of openings or through apertures 12 in guide plate 10 in FIG. 1. The ultrasound system can be associated with or contain a dosimetry program used to calculate the location of the radioactive seeds to be placed in the prostate, as is known in the art. An electronically generated grid of the type illustrated in the template or guide plate 10 of FIG. 1 is generally superimposed on the images to assist in visualizing positions of features on the images, or to guide the clinician in biopsy, brachytherapy and cryosurgery for cancerous prostate and related surgeries.

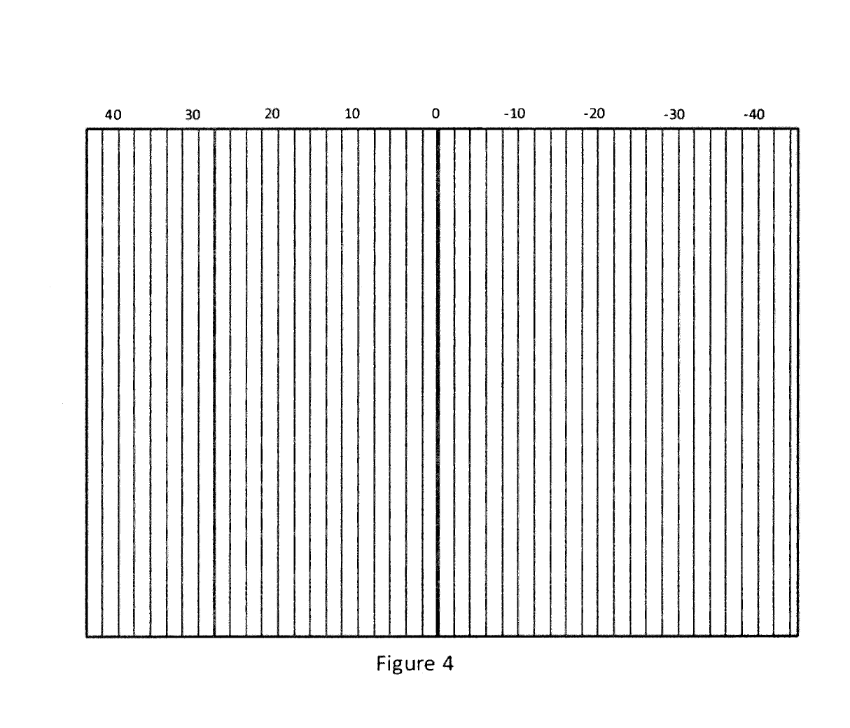

An alternative grid to be electronically generated for superposition on the displayed images is illustrated in FIG. 4. This grid may be especially useful for displaying the slice of the image that is displayed in the sagittal or transverse view.

Referring to FIG. 3, and as discussed in U.S. Pat. No. 6,752,753, a conventional brachytherapy system including a cradle 300, has a stepping unit 302. One or more needles 306 are guided into the patient by the cradle 300 as the needles 306 are moved by the stepping unit 302. Another example of a conventional stepping unit is disclosed in U.S. Pat. No. 5,871,448. The manner in which seeds are placed using this type of apparatus is discussed in detail in U.S. Pat. No. 6,752,753, and is incorporated herein by reference.

An ultrasound probe 310 coupled to the stepping unit 302 is inserted rectally to a depth adjacent a portion of a patient's prostate 321. The ultrasound probe 310 emits waves directed through the adjacent portion of the prostate 321 from which an ultrasonic cross-sectional image of that portion of the prostate 321 can be obtained. The stepping unit 302 incrementally moves the ultrasound probe to obtain ultrasound images of successive planes of the prostate or other organ or tissue. The stepper unit 302 includes a scale 311 oriented in the z direction that shows the depth of the ultrasonic probe 310 relative to a predetermined datum.

Ultrasound probe 310 containing an accelerometer or other type of sensor 325 such as a magnetometer or gyroscope that can be used individually or in combination and that is coupled to an ultrasonic imaging system 309 by a cable 313, or by a radio frequency link, such as Wi-Fi. System 309 and probe 310 may be of the kind described in United States Patent Publication 20040152986, useful for transverse and sagittal image acquisition. System 309 includes an electronics module, the module having: excitation circuitry for exciting probe 310, receiving circuitry for processing signals received from probe 310, signal processing circuitry for processing signals from the receiving circuitry to produced processed image signals, software to compute the probe position from the embedded sensor information; and at least one display 307 for displaying the processed image signals. Display 307 can be at least a two part display having a first portion for displaying ultrasound images and a second portion for displaying images obtained by using a different imaging modality.

Referring also to FIG. 2, in the illustrated embodiment, a first display portion 307A displays an MRI image 201 (discussed below). A second display portion 307B displays a transverse ultrasound image 202 obtained by the system 300. A third display portion 307C displays a sagittal ultrasound image 204, having a target area 203. The image 204 is also obtained from the system 300.

Each of display portions 307A, 307B and 307C can be thought of as a separate display. All three displays can be parts of a single display screen. Alternatively, separate display screens or monitors, located in proximity to one another, for easy simultaneous viewing by the clinician, can be used. Thus, each of display portions 307A, 307B and 307C can be thought of a separate display. At least two separate displays, one for each image modality being used, are preferable, with three being used in the embodiment described herein. While much less convenient, the two or more images being displayed can be successively toggled on a single display screen.

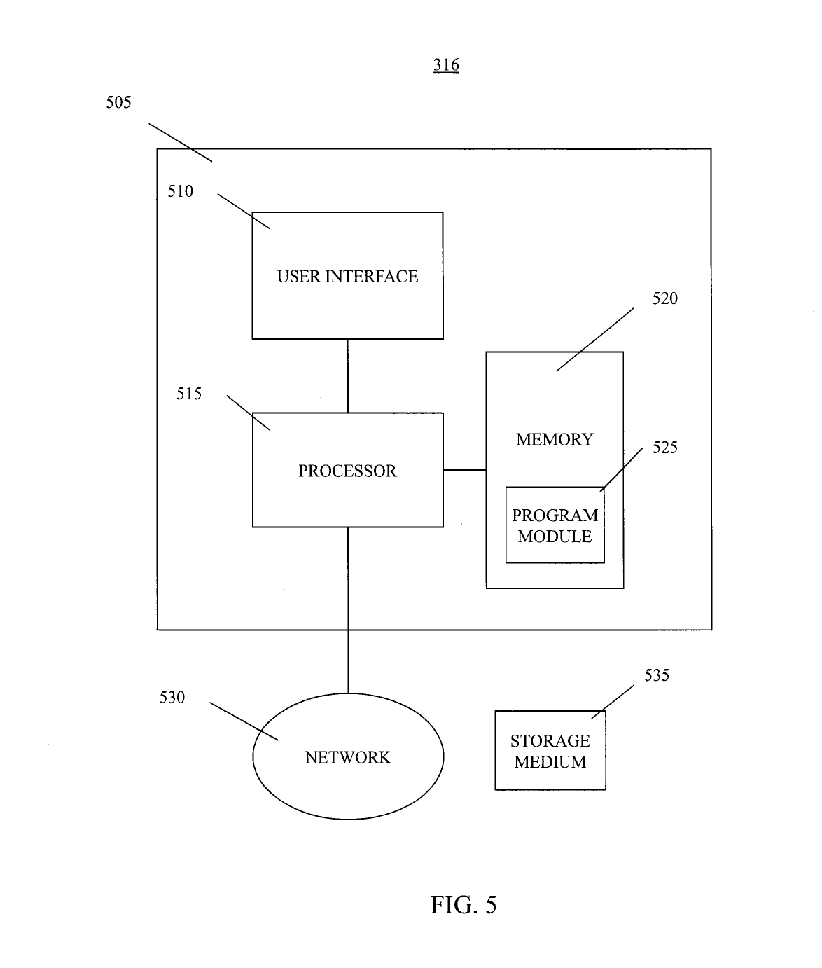

A computer system 316, described in detail in FIG. 5 herein, is associated with ultrasonic imaging system 309. Images data from ultrasonic imaging system 309 is supplied to computer system 316 by a cable 315, which may be a USB cable. A cable 311 interfaces to template or guide plate 10 to control the indications of probe or needle placement on the representations on the display screens, of openings 12, as described above.

An input device 330 for receiving second images includes an image data acquisition apparatus. Input device 330 may be any device that provides image data to computer 316. Thus, it may be a drive for accepting a magnetic medium, a CD or a DVD. Alternatively, input device 330 may be a network connection device, such as a connector to a network card in computer 316. An input device 330 can also be a Digital Imaging and Communications in Medicine (DICOM) capable device, as described below.

An MRI image set or other modality, displayed as image 201 is imported to the system from a CD or other image storage source via input device 330. The image slices can be displayed and scrolled through on the left image on the screen as image 201. As noted above, the images may be stored on and acquired from a Digital Imaging and Communications in Medicine (DICOM) device, a standard for handling, storing, printing, and transmitting information in medical imaging.

A corresponding data set is collected with the ultrasound system by placing the transverse array of the transducer so that it images the base of the prostate, and pulling the transducer slowly out of the rectum to automatically collect, at spaced intervals, a series of images (or data set) matching the MRI data. Since the resolution of typical imaging systems is approximately 2 mm, the data set is typically comprised of slices taken at 2 mm intervals. Any one of these slices can be displayed. The corresponding slice, acquired by MRI or another imaging modality, is also displayed, preferably simultaneously, as described above. An anatomical target, such as the base of the prostate gland, is located, as a reference point for the operator to pull the transducer further out of the rectum while acquiring the images at intervals.

The accelerometer sensor, or other sensor 325, as described above, can be added to a standard ultrasonic imaging system, which has software added thereto, and input for receiving data from sensor reading module, to assemble a system in accordance with the disclosure. Thus, existing imaging systems may readily be adapted to implement the presently disclosed invention, as described herein.

Thus, using the apparatus and method of the invention, the treating physician can simultaneously view the same image plane in both imaging modalities.

As noted above, imaging system 309 may have a software module 320 which assists in determining the position of the transducer in the transverse plane.

FIG. 5 is a block diagram of computer system 316, for employment with the embodiments described herein. System 316 includes a computer 505 coupled to a network 530, e.g., the Internet.

Computer 505 includes a user interface 510, a processor 515, and a memory 520. Computer 505 may be implemented on a general-purpose microcomputer. Although computer 505 is represented herein as a standalone device, it is not limited to such, but instead can be coupled to other devices via network 530.

Processor 515 is configured of logic circuitry that responds to and executes instructions, such as those in software module 320.

Memory 520 stores data and instructions for controlling the operation of processor 515. Memory 520 may be implemented in a random access memory (RAM), a hard drive, a read only memory (ROM), or a combination thereof. One of the components of memory 520 is a program module 525.

Program module 525 contains instructions for controlling processor 515 to execute the methods described herein. For example, as a result of execution of program module 525, processor 515 can facilitate the simultaneous display of images obtained by using two different imaging modalities. The term “module” is used herein to denote a functional operation that may be embodied either as a stand-alone component or as an integrated configuration of a plurality of sub-ordinate components. Thus, program module 525 may be implemented as a single module or as a plurality of modules that operate in cooperation with one another. Moreover, although program module 525 is described herein as being installed in memory 520, and therefore being implemented in software, it could be implemented in any of hardware (e.g., electronic circuitry), firmware, software, or a combination thereof.

User interface 510 includes an input device, such as a keyboard or speech recognition subsystem, for enabling a user to communicate information and command selections to processor 515. User interface 510 also includes an output device such as a display or a printer. A cursor control such as a mouse, track-ball, or joy stick, allows the user to manipulate a cursor on the display for communicating additional information and command selections to processor 515.

Processor 515 outputs, to user interface 510, a result of an execution of the methods described herein. Alternatively, for use in the system and method disclosed herein, processor 515 directs the output to a remote device, in this case ultrasound system 309 via cable 315 (or via network 530) to display the images obtained by different imaging modalities on display 307 and its portions 307A, 307B and 307C.

While program module 525 is indicated as already loaded into memory 520, it may be configured on a storage medium 535 for subsequent loading into memory 520. Storage medium 535 can be any conventional storage medium that stores program module 525 thereon in tangible form. Examples of storage medium 535 include a floppy disk, a compact disk, a magnetic tape, a read only memory, an optical storage media, universal serial bus (USB) flash drive, a digital versatile disc, or a zip drive. Alternatively, storage medium 535 can be a random access memory, or other type of electronic storage, located on a remote storage system and coupled to computer 505 via network 530.

It will be understood if the image data acquired by using more than one modality is supplied to computer 316 from an external source, and more than one display portion is provided, the image data may be compared without the use of ultrasound system 309.

It also will be understood that best result in terms of ease of use and interpretation of the images will be obtained if the image slices for both the first series of images acquired using a first imaging modality, and the second series of images acquired using the second imaging modality are along the same line or axis with respect to the body of the patient (or at the very least along axes that are substantially parallel to one another). Further, the slices for both the first series of images acquired using the first imaging modality, and the second series of images acquired using the second imaging modality should be obtained with uniform spacing from one another.

The apparatus and method disclosed herein advantageously can be used for both diagnosis and treatment. The ability to view the same slice of an organ in using two different imaging modalities, provides the diagnostician with more information than either modality individually could possibly provide. Pathology which could possibly not be visible in an imaged produced by using a first modality may be visible in an image produced using a second modality. One of the modalities may be better suited to assist in treating the patient. For example, ultrasound may be more suitable than other modalities in assisting in biopsy, brachytherapy and cryosurgery for a cancerous prostate, and in related surgeries

It will be understood that the invention may be embodied in a computer readable non- transitory storage medium storing instructions of a computer program which when executed by a computer system results in performance of steps of the method described herein. Such storage media may include any of those mentioned in the description above.

The techniques described herein are exemplary, and should not be construed as implying any particular limitation on the present disclosure. It should be understood that various alternatives, combinations and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “comprises” or “comprising” are to be interpreted as specifying the presence of the stated features, integers, steps or components, but not precluding the presence of one or more other features, integers, steps or components or groups thereof.

What is claimed is:

1. An apparatus for displaying images of an organ within the body, comprising:
an ultrasonic imaging system having at least one display screen;
an ultrasonic transducer associated with the ultrasonic imaging system, the ultrasonic transducer being movable to acquire first images of first successive slices of the organ for display by the at least one display screen of the imaging system;
a position sensor associated with the ultrasonic transducer for sensing the position of the ultrasonic transducer when the first images of the first successive slices are acquired; and
an input for receiving second images of second successive slices of the organ generated by a second imaging system other than an ultrasonic imaging system, the second successive slices of the organ being sequentially displayed on the at least one display screen;
wherein one of the first successive images and one of the second successive images, each corresponding to a same slice of the organ, are simultaneously displayed on the at least one display screen.

2. The apparatus of claim 1, wherein the input for receiving second images includes an image data acquisition apparatus.

3. The apparatus of claim 2, wherein the image data acquisition apparatus acquires data from an image data storage medium.

4. The apparatus of claim 3, wherein the image storage medium is selected from the group consisting of a CD, a DVD, a magnetic disk drive and a flash memory.

5. The apparatus of claim 2, wherein the image data acquisition apparatus acquires data from a DICOM device.

6. The apparatus of claim 1, wherein the second imaging system is an MRI system.

7. The apparatus of claim 1, wherein the second imaging system is on selected from the group consisting of an x-ray imaging system and a computerized axial tomography imaging system.

8. The apparatus of claim 1, wherein the position sensor is an accelerometer, and position of the ultrasonic transducer is determined by integrating signals from the accelerometer.

9. An apparatus for displaying images of an organ within a body comprising:
at least one display screen for displaying an image of a first slice of an organ obtained using a first imaging modality; and
the at least one display screen also displaying an image of a second slice of an organ obtained using a second imaging modality;
wherein the first slice and the second slice are substantially the same slice of the organ.

10. The apparatus of claim 9, wherein the first slice is one of a first series of successive slices obtained by the first modality and the second slice is one of a second series of slices obtained by the second modality.

11. The apparatus of claim 9, wherein the first slice and successive ones of the series of slices obtained by the second modality are displayed on the at least one display screen.

12. The apparatus of claim 9, wherein the first imaging modality is ultrasound.

13. The apparatus of claim 9, wherein the second imaging modality is MRI.

14. The apparatus of claim 9, wherein the second imaging modality is selected from the group consisting of x-ray imaging and computerized axial tomography imaging.

15. The apparatus of claim 9, wherein first series of successive slices obtained by the first modality and the second series of slices obtained by the second modality are perpendicular to substantially the same axis.

16. A method for displaying images of an organ within the body, comprising:
displaying on at least one display screen, an image of a first slice of an organ obtained using a first imaging modality; and
displaying on the at least one display screen, an image of a second slice of an organ obtained using a second imaging modality;
wherein the first slice and the second slice are substantially the same slice of the organ.

17. The method of claim 16, wherein the first slice is one of a first series of successive slices obtained by the first modality and the second slice is one of a second series of slices obtained by the second modality.

18. The method of claim 16, further comprising displaying on the at least one display screen the first slice and successive ones of the series of slices obtained by the second modality.

19. The method of claim 16, wherein the first imaging modality is ultrasound.

20. The method of claim 16, wherein the second imaging modality is MRI.

21. The method of claim 16, wherein the second imaging modality is selected from the group consisting of x-ray imaging and computerized axial tomography imaging.

22. The method of claim 16, wherein the first series of successive slices obtained by the first modality and the second series of slices obtained by the second modality are perpendicular to substantially the same axis.

23. The method of claim 16, wherein the first modality is ultrasonic imaging, the method further comprising:
using a movable ultrasonic transducer associated with the ultrasonic imaging system to acquire the first images of first successive slices of the organ for display by the at least one display screen of the imaging system; and
acquiring a position signal representative of position of the sensor associated with the ultrasonic transducer to sense the position of the ultrasonic transducer when the first images of the first successive slices are acquired.

24. A computer readable non-transitory storage medium storing instructions of a computer program which when executed by a computer system results in performance of steps of a method for imaging comprising the steps of:
displaying on at least one display screen, an image of a first slice of an organ obtained using a first imaging modality; and
displaying on the at least one display screen, an image of a second slice of an organ obtained using a second imaging modality;
wherein the first slice and the second slice are substantially the same slice of the organ.
